(19) United States
(12) Patent Application Publication
(10) Pub. No.: US 2006/0184082 A1
(43) Pub. Date: Aug. 17, 2006

(54) TRACTION DEVICE

(75) Inventors: Christian HUNT, McLean, VA (US); Ara BOGHOSIAN, Alexandria, VA (US); John A. SPADAVECCHIA, Red House, VA (US)

Correspondence Address: GREENBLUM & BERNSTEIN, P.L.C., 1950 ROLAND CLARKE PLACE, RESTON, VA, 20191, US

(73) Assignee: CARE REHAB & ORTHOPEDIC PRODUCTS, INC., McLean, VA (US)

(21) Appl. No.: 11/275,971

(22) Filed: Feb. 7, 2006

Related U.S. Application Data
(60) Provisional application No. 60/653,106, filed on Feb. 16, 2005.

(51) Int. Cl. A61F 5/00

(52) U.S. Cl. 602/32; 602/36

(57) ABSTRACT

A device includes a stationary housing. A carriage is slidable mounted on the housing and at least one corrugated diaphragm mechanism is housed in the housing and connectable to the carriage. The corrugated diaphragm is structured to elastically expand and retract, depending on an application of pressure, to extend and retract, respectively, the carriage relative to the stationary housing.

### CROSS REFERENCE TO RELATED APPLICATIONS

This application claims priority to U.S. Provisional Application Ser. No. 60/653,106, filed on Feb. 16, 2005, the contents of which are incorporated in its entirety herein.

### FIELD OF THE INVENTION

The invention generally relates to a cervical and/or lumbar traction device, and more particularly, to a cervical and/or lumbar traction device having a diaphragm device for slidably moving a carriage.

### DISCUSSION OF BACKGROUND INFORMATION

Traction devices are used to relieve pressure on inflamed or enlarged nerves. Cervical and lumbar or spinal traction devices are the most common type of these devices. When correctly used, the traction devices can relieve pain in the neck and the spine by, for example, straightening the curvature of the spine or stretching of the spinal and cervical musculature,

Portable traction devices are now becoming very popular for in home use. These devices allow patients to perform traction therapy without leaving their homes, or expending large sums of money for a healthcare provider or physical therapist. Under the proper guidance and instruction, these portable devices are becoming ever more common, especially in today's age of rising health care costs.

Additionally, known portable lumbar traction devices, currently manufactured and sold, are cumbersome and difficult to transport. These lumbar traction devices are basically a large board of approximately 3 or more feet in length.

In one known portable lumbar traction device, two flat separate boards are assembled in order to form the platform for the device. To make such assembly, hooks extending from one flat board are aligned with holes in the other flat board. This is accomplished most typically, by lifting and aligning the board with the hooks and then inserting the hooks into the holes. During this assembly, a piston rod of a pneumatic device remains attached to one board and the cylinder housing of the pneumatic device remains attached to the other board , making for a very awkward assembly. To disassemble the traction device, the hooks must be removed from the holes, which is an awkward process, especially in view of the piston rods and cylinders remaining attached between the separated boards.

### SUMMARY OF THE INVENTION

In a first aspect of the invention, a device includes a stationary frame and a carriage slidable mounted on the frame. At least one corrugated diaphragm mechanism is connectable to the carriage The corrugated diaphragm is structured to elastically expand and retract, depending on an application of pressure, in order to extend and retract, respectively, the carriage.

In another aspect of the invention, the device includes a frame having a housing for accommodating at least one corrugated diaphragm device which is expandable and retractable by application and release of pressure, respectively. The at least one corrugated diaphragm device has a retractable spring force such that, upon release of pressure within the at least one corrugated diaphragm device, the at least one corrugated diaphragm device begins to retract moving the carriage towards an initial position.

In another aspect of the invention, the device includes a stationary frame having a housing for accommodating at least one corrugated diaphragm device which is expandable and retractable by application and release of pressure, respectively. The at least one corrugated diaphragm device is mounted to a moveable carriage which is slidable along the stationary frame between an initial position and a fully extended position. An overall length of the device remains constant upon the application of pressure.

### BRIEF DESCRIPTION OF THE DRAWINGS

The present invention is further described in the detailed description which follows, wherein:

FIG. 1 shows a top view of a cervical traction device in accordance with the invention;

FIGS. 2a and 2b show side views of the cervical traction device in an extended and retracted position, respectively, in accordance with the invention;

FIG. 3 shows a cut-away view of the cervical traction device in 4 accordance of the invention;

FIG. 4 shows an exploded view of the cervical traction device in accordance with the invention;

FIGS. 4a and FIG. 4b (cross section) show a connection mechanism in accordance with the invention;

FIG. 5 shows a bottom view (with a bottom housing removed) of the cervical traction device in accordance with the invention;

FIG. 5a shows views (with cushion wedges removed) of the cervical traction device in accordance with the invention;

FIG. 6 shows a lumbar traction device in accordance with the invention; and

FIG. 7 shows an exploded view of the lumbar traction device in accordance of the invention.

### DETAILED DESCRIPTION OF EMBODIMENTS OF THE INVENTION

The invention is directed to a cervical and/or a lumbar traction device. The traction device includes a corrugated diaphragm which is structured and adapted to move a carriage in a linear fashion. The diaphragm includes a certain spring force which is capable, upon the release of pressure therein, to move the carriage towards its original position and, in embodiments, substantially to its original position. In addition to its own spring force, a roll spring or other spring or resilient type mechanism may be used in combination with the diaphragm to retract the carriage towards its original position. The diaphragm is devoid of gaskets and other moving parts. The cervical and/or a lumbar traction device, using the diaphragm and related components, is lighter and includes less parts than devices using pneumatic cylinders.

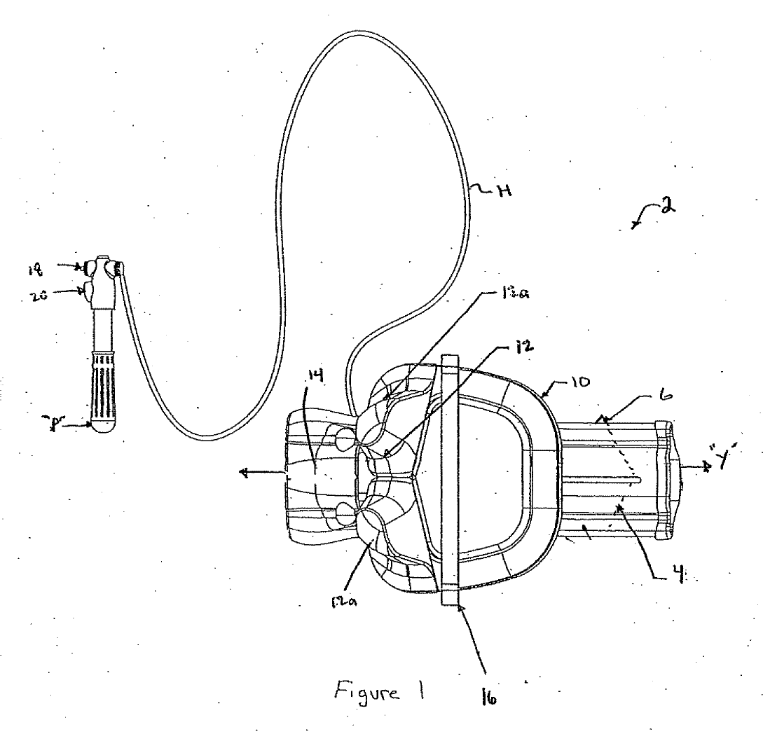

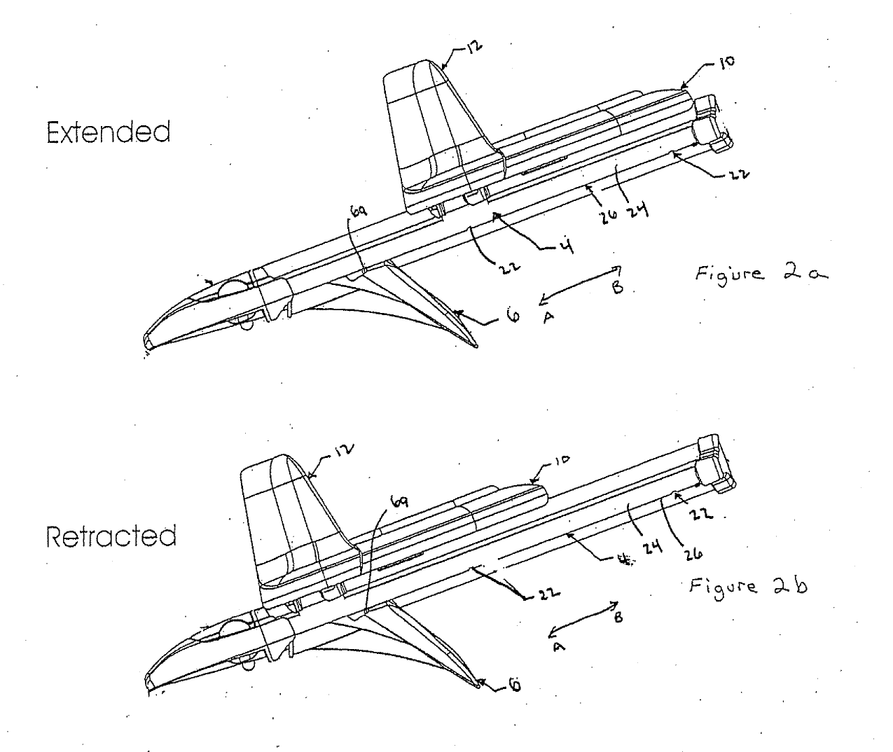

FIG. 1 shows an embodiment of the cervical traction device. The cervical traction device is generally denoted as reference numeral 2. The traction device 2 includes a stationary housing (e.g., frame) 4 having a moveable stand 6 which is structured and adapted so that several angles can be achieved relative to a flat surface, e.g., a floor or other surface where a user may use the device 2. The cervical traction device 2 further includes a movable headrest 10 having a occiput wedge system 12, which is mounted to a sliding carriage 14. A strap or other restraining device 16 for restraining a user's head is attached to the movable headrest system 10.

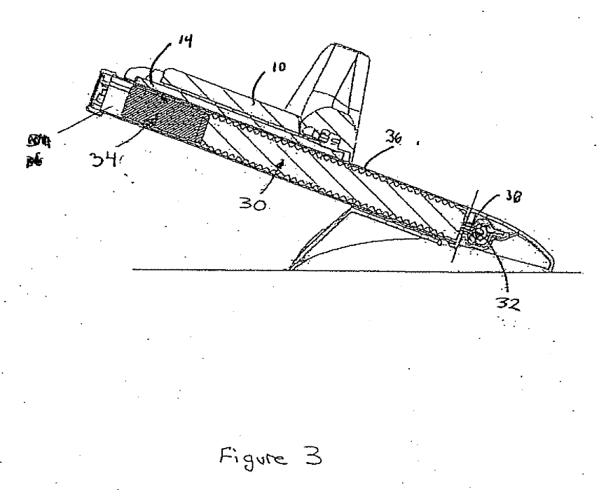

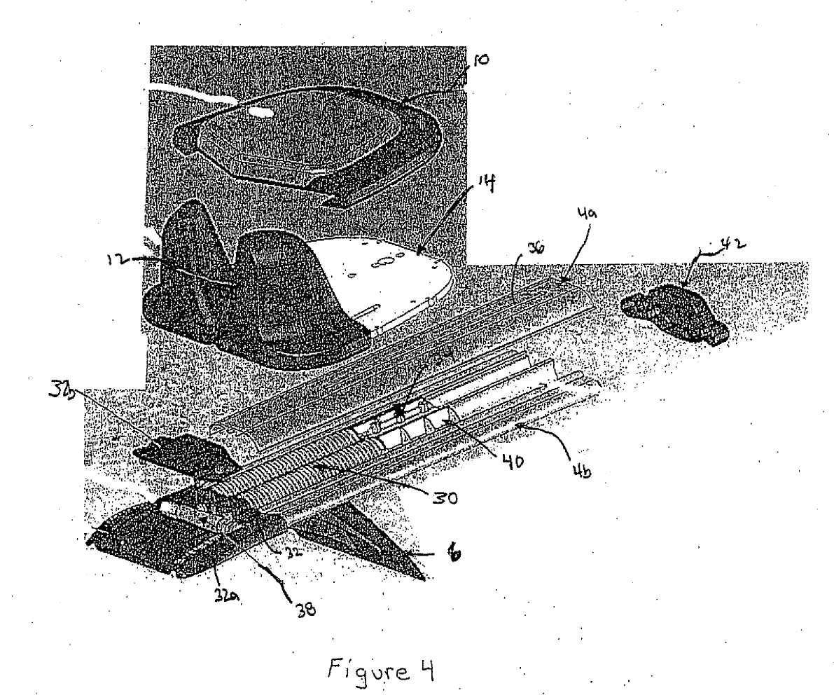

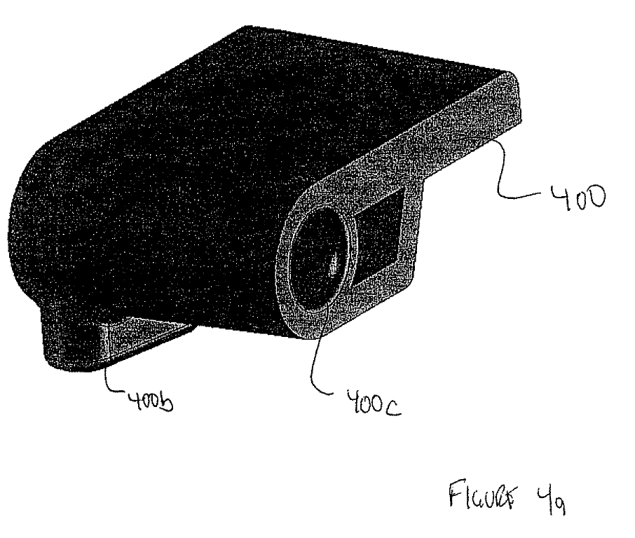

The occiput wedge system 12 includes separate wedges 12a that may have a concave engaging surface. The occiput wedge system 12, with the headrest 10, may be slidably movable along a longitudinal axis “Y” of the housing 4 by a corrugated diaphragm type device (shown more clearly in FIG. 3). The occiput wedge system 12 is designed to apply a therapeutic traction force to the occipital areas on a patent's head, while the housing 4 remains stationary. This is accomplished, in one aspect of the invention, by pressure being applied by a pump P via a hose H, to the diaphragm type device. Thus, upon pressurization, the corrugated diaphragm type device will move or slide the carriage 14 and thus the headrest 10 and occiput wedge system 12. The pump P may have a manual pressure relief mechanism 18, as well as a gauge 20 to show a force created on within the diaphragm type device.

FIGS. 2a and 2b show the cervical traction device 1 positioned at an angle of, for example, 10 degrees relative to a flat surface, e.g., a floor or other surface where the user may use the device 2. It should be understood that other angles of adjustment, e.g., 15 degrees, 20 degrees, etc. with relation to the supporting surface, are also contemplated by the invention, and that the angles shown herein are provided for illustrative purposes. In the examples of FIGS. 2a and 2b, the adjustment can be accomplished via movement of the moveable stand 6 in either direction “A” or “B”, along the longitudinal axis of the device 2.

Still referring to FIGS. 2a and 2b, the housing 4 includes a guide 24 which has turned ends that form rails 26. The rails 26 are structured to accommodate the stand 6 In one embodiment, the rails 26 include a plurality of notches 22 for locking or positioning of the stand 6 at a predetermined position. The notches 22 may also be apertures or holes. In embodiments, the stand 6 may be locked into place by use of a spring arm, hook or other type of mechanism 6a that engages the notches 22 positioned along the stand 6 of the housing 4.

As discussed in greater detail below, in FIG. 2a, the traction device is in an extended position; whereas, in FIG. 2b, the traction device is in a retracted position. The extended position is achievable by pressurizing the diaphragm shown in FIG. 3. The retracted position is achievable, at least in part, by a return spring force of the diaphragm. That is, upon release of pressure, the spring force of the diaphragm is capable of moving the carriage towards its original position and, in embodiments, substantially to its original position.

FIG. 3 shows a cutaway view of the traction device in accordance with the invention. The traction device 2 includes a corrugated diaphragm type device 30 that is mounted within or mounted to the housing 4. In embodiments, the corrugated diaphragm type device 30 is mounted to a support structure 32 of the housing 4 and the movable carriage 14. In one implementation, at least one boss 34 extends from the moveable carriage 14, via a slot 36 extending substantially along a length of the housing 4, into an interior space of the housing 4, which is connected to a mounting carriage 35. In this embodiment, the corrugated diaphragm type device 30 is attached to the at least one boss 34 via a mounting carriage 35 adapted and configured to slide within the housing 4 and preferably a profile 35a of the housing 4 that is configured to house the corrugated diaphragm type device 30. Upon pressurization of the corrugated diaphragm type device 30, the corrugated diaphragm type device 30 will move the carriage 14 to apply a traction force

The corrugated diaphragm type device 30 is connected to an air inlet or manifold 38 provided at an end of the housing 4, proximate to the support structure 32. The manifold 38 is connected to one or more of the diaphragm type devices 30 and is structured to allow pressurized air from the pump “P” to pass into the corrugated diaphragm type device 30.

Still referring to FIG. 3, the corrugated diaphragm type device 30 acts as a spring, e.g., has its own spring force, to retract the carriage in both a cervical and lumbar traction system from an expanded position. In one exemplary illustration, the corrugated diaphragm type device 30 can be expanded to approximately 10.5 inches in the expanded position, and in one embodiment may have a starting position of about 6.5 inches in length (with an original neutral length of approximately 8 inches). It is also contemplated that other starting and expanded positions are provided by the invention. In one implementation, a stroke length of the corrugated diaphragm type device 30 is approximately 4 inches; although other stroke lengths are also contemplated by the invention.

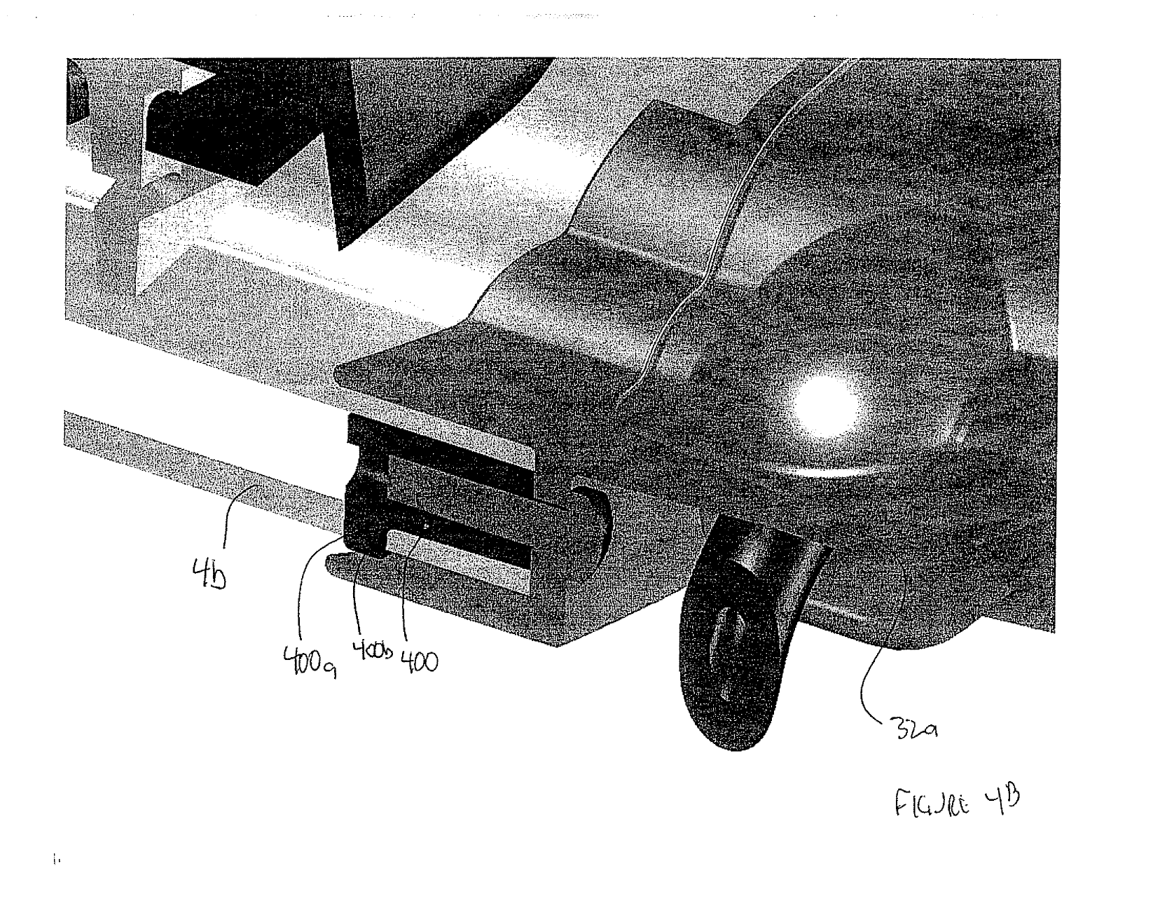

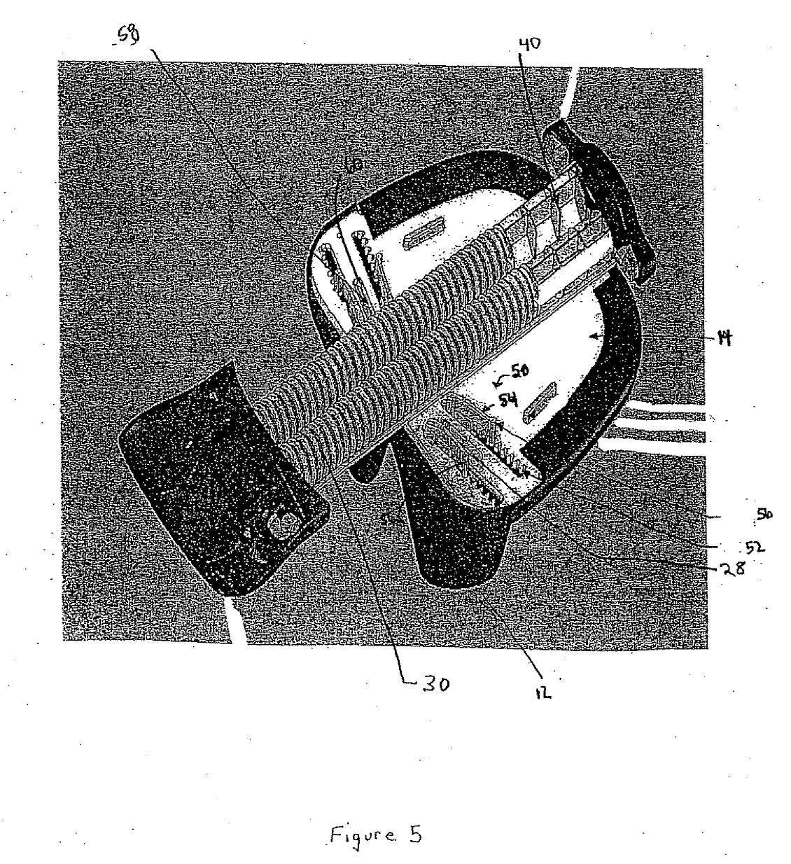

FIG. 4 shows an exploded view of the traction device in accordance with the invention. The traction device includes, for example, the headrest 10 and occiput wedge system 12 connected to the carriage 14. The carriage 14, in turn, is connectable to the at least one boss 34 which extends through the slot 36 along the housing 4 and more specifically a cover plate 4a of the housing 4. The housing 4 further includes a lower frame portion 4b, which includes an interior portion 40 for housing the corrugated diaphragm type device 30.

The housing 4 further includes the support structure 32, which may include an extending portion 32a for resting on a supporting surface. A cover 32b may be used, in conjunction with the extending portion 32a, to form an interior space, which accommodates the manifold 38. An end cap 42 may be connected to the housing 4, opposing the extending portion 32a. Thus, the combination of the end cap 42, top plate 4a, bottom frame 4b and support structure forms an interior housing for the corrugated diaphragm type device 30. In alternate embodiments, the corrugated diaphragm type device 30 is mounted or attached to the mounting carriage 35 which has at least one extending boss 34 adapted and configured to slide within a guide of the housing 4 and connectable to the sliding carriage 14.

The extending portion 32a may be held in place by a nut 400 having a projection 400b, as shown in FIGS. 4a and 4b. In this implementation, the nut will be bolted via the hole 400c and the projection 400b will communicate with a slot 400a in the frame portion to prevent rotation of the extending portion 32a and to ensure a connection therebetween Alternatively, the extending portion 32a may be connected or mated with the frame by any known, conventional manner such as a screw, nut and bolt system, etc.

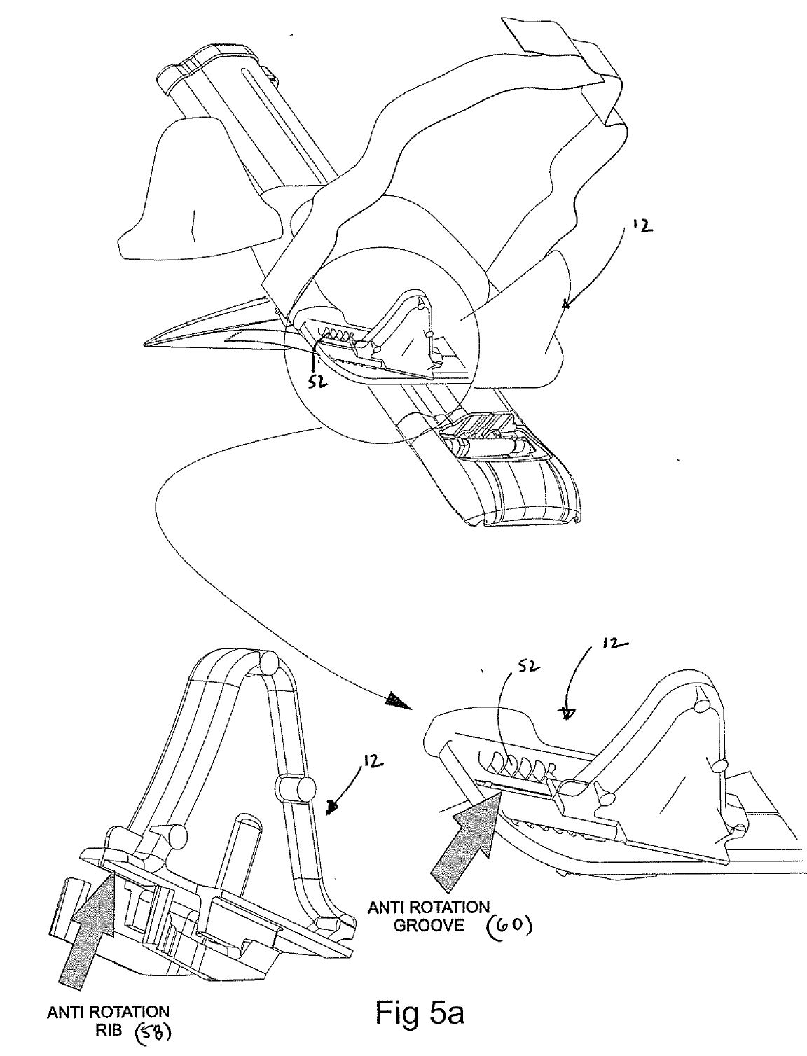

FIG. 5 shows a bottom view (with a bottom portion of the housing removed) of the traction device 2 in accordance with the invention. In this example, the wedges 12 are designed to move in a lateral direction, e.g., do not rotate. In this embodiment, the wedges 12 can be adjustable by a pinch spring arm mechanism 50. The pinch spring arm mechanism 50 includes a ratchet type guide 52 in/on the moveable carriage 14. The pinch spring arm mechanism 50 further includes spring arms 54 which may engage the ratchet guide 52 at different positions via opposing protruding portions 56. The opposing protruding portions 56 have tabs 56a so that the user can squeeze them together to disengage from a ratchet portion of the ratchet guide 52, and then move the wedges in an inward position or an outward position. The movement of the wedges may be in a somewhat linear manner across the carriage 14.

Still referring to FIG. 5 and additionally FIG. 5a, the wedge system 12 may also include an anti-rotation rib 58 and corresponding slot or groove 60 for preventing rotation of the wedge 12. The slot 60 can be part of the carriage 14 and is, in one implementation, proximate to the ratchet guide 52. The rib 58 communicates with the slot 60 while allowing the wedges 12 to slide in and out.

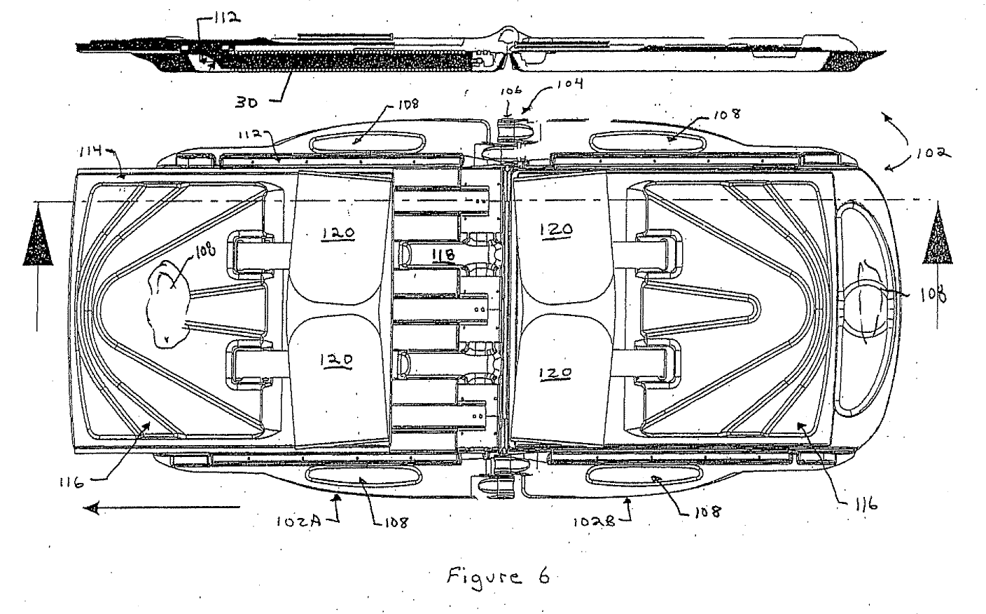

FIG. 6 shows an embodiment of a lumbar traction device 100 in accordance with the invention. Similar to the traction device shown and described with reference to FIGS. 1-5, a corrugated diaphragm type device is used to apply a traction type force. In the lumbar traction device 100, three corrugated diaphragm type devices may also be used to provide the traction force; although other numbers of the corrugated diaphragm type devices are also contemplated for use with the invention. Integrally molded frame members 102A and 102B are hingedly attached via a hinge type mechanism 104 (which may be an alternating type hinge mechanism having at least four parts) to form a portable, foldable device. In the closed position, for example, the frame members 102A and 102B face one another; whereas, in the open state, the frame members 102A and 102B extend along a longitudinal axis in a single plane. Thus, the design of the traction device of FIG. 6 allows for the easy closing and opening of the lumbar traction device 100 without the requirement for aligning any parts, lifting any of the parts or separating any of the parts. The lumbar traction device 100 also includes integrally formed handles 108, for example, molded on sides or the ends thereof.

Still referring to FIG. 6, the frame members 102A and 102B are designed to house or mount thereon many of the components of the lumbar traction device 100, such as the corrugated diaphragm type device 30. By way of illustration, the lumbar traction device 100 may include guides 112 positioned on opposing sides of the frame member 102 and/or frame member 102B. The guides 112 are designed to seat thereon a slidable lumbar carriage 114. The lumbar carriage 114 is slidably moveable along the longitudinal axis of the frame 102A and 102B to provide a traction force such that, upon pressurization or release thereof, the corrugated diaphragm type device moves the slidable lumbar carriage 114 in directions “A” and “B”. In one embodiment, the lumbar carriage 114 includes a support pad 116 for the comfort of a user. A pair of belts or other restraining device 120 can be mounted to the support pad 116 and/or the frame member(s). The slidable lumbar carriage 114 may also be seated within a recessed portion 118 of the frame.

Referring to FIG. 71 the slidable lumbar carriage 114 is connected to the corrugated diaphragm type device 30 in order to provide a traction force. Much like discussed above, the corrugated diaphragm type device 30 is mounted within a housing or recess 122 formed by the frame member and a cover plate 124. In embodiments, the lumbar traction device may include two or three corrugated diaphragm type devices 30, corresponding to the number of recesses and/or the required application of traction force.

The corrugated diaphragm type device 30 is connected to a manifold 126 which, in turn, is mounted to a support structure 128. The manifold 126 includes an air intake/exhaust 132 connected to a hose “H” of the hand pump “P”. The corrugated diaphragm type device 30 is also connected to the slidable lumbar carriage 114 via a boss 134. In one implementation, the boss 134 extends from the slidable lumbar carriage 114, via s slot or cutout 136 extending substantially along a length of the plate 124. In this embodiment, the corrugated diaphragm type device 30 is coupled to the boss 134 and, upon pressurization of the corrugated diaphragm type device(s) 30, will move the slidable lumbar carriage 114 to apply a traction force. The support pad 1 16 may be mounted to the slidable lumbar carriage 114 via a plate 114a (and fasteners, e.g., screws, rivets, etc.)

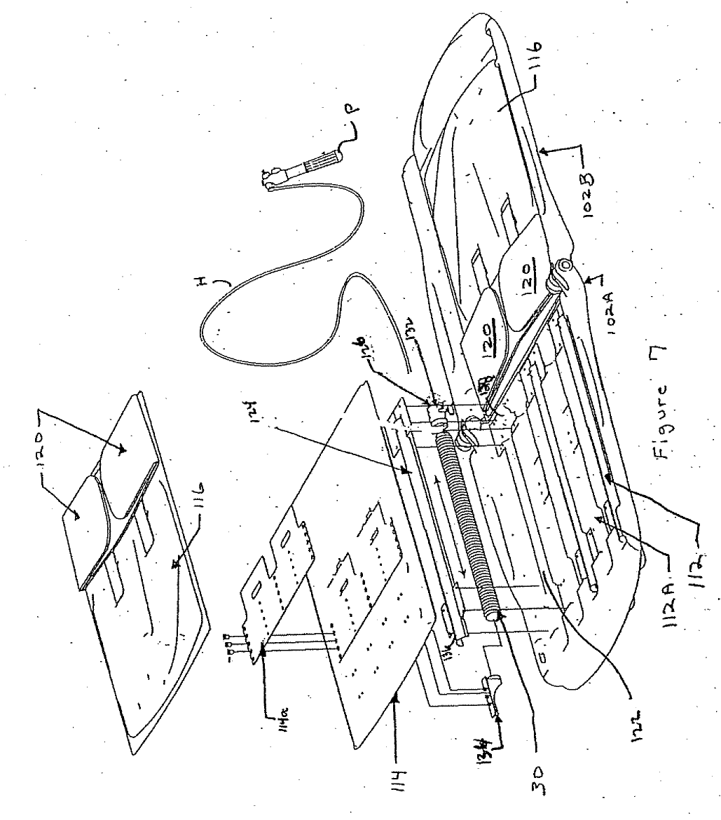

Still referring to FIG. 7, the corrugated diaphragm type device 30 acts as a spring, e.g., has its own spring force, to retract the carriage in both a cervical and lumbar traction system from an expanded position. In addition to its own spring force, a roll spring or other spring or resilient type mechanism may be used in combination with the diaphragm, to retract the carriage towards its original position which may be represented schematically with the diaphragm. In one exemplary illustration, the corrugated diaphragm type device 30 can be expanded to approximately 10.5 inches in the expanded position, and in one embodiment may have a starting position of about 6.5 inches in length. It is also contemplated that other starting and expanded positions are provided by the invention. In one implementation, a stroke length of the corrugated diaphragm type device 30 is approximately 4 inches; although other stroke lengths are also contemplated by the invention.

### Operation of the Traction Device

To use the traction devices, the corrugated diaphragm type device 30 is initially at atmospheric pressure. With a cervical traction device, prior to use, the patient or user can adjust the angle of the cervical traction device by adjusting the positioning of the stand. The back of the user is placed on a support surface so that the wedges cradle the patient's neck. The wedges can also be adjusted but preferably remain substantially stationary during use of the device. The restraining device may be used about the patent's head to ensure the user remains substantially stationary during treatment of cervical traction device. In the lumbar device, the patient would strap the restraining device about his/her mid section.

Once properly positioned, the patient then pumps air into the corrugated diaphragm type device. The patient increases the traction force by manually operating the pump or decreases the traction force by manually pressing the pressure relief mechanism. As air is pumped into the corrugated diaphragm type device, the corrugated diaphragm type device will expand and move the carriage to apply a traction force. The entire structure of the cervical traction device, though, remains constant, during the application of the traction force,

While the invention has been described in terms of embodiments, those skilled in the art will recognize that the invention can be practiced with modification.

What is claimed:

1. A device, comprising:
a stationary frame;
a carriage slidable mounted on the stationary frame; and
at least one diaphragm mechanism connectable to the carriage, the diaphragm mechanism is structured to elastically expand and retract, depending on an application of pressure, respectively, and to move the carriage relative to the stationary frame.

2. The device according to claim 1, wherein the at least one diaphragm is a corrugated diaphragm.

3. The device according to claim 1, wherein the at least one diaphragm has a spring force such that upon release of pressure the at least one diaphragm retracts and moves the carriage toward an initial position.

4. The device according to claim 1, wherein the at least one diaphragm has an approximate length of 10.5 inches in the extendable position.

5. The device according to claim 4, wherein the at least one diaphragm has an approximate length of 6.5 inches at a starting position.

6. The device according to claim 1, wherein a stroke length of the at least one diaphragm from a starting position to an extendable position is approximately 4 inches.

7. The device according to claim 1, wherein the at least one diaphragm is in communication with a manifold in communication with a pump system.

8. The device according to claim 7, wherein the pump system includes a pressure relief mechanism to manually release the pressure in the at least one diaphragm and a gauge indicating an amount of pressure in the at least one diaphragm.

9. The device according to claim 1, wherein the at least one diaphragm is two or three diaphragms.

10. The device according to claim 1, wherein the at least one diaphragm is mounted between a portion of the frame and a portion of the carriage.

11. The device according to claim 1, further comprising at least one restraining mechanism to releasably restrain a portion of a person's body to the slidable carriage.

12. The device according to claim 1, wherein the device is a cervical traction device or a lumbar traction system.

13. The device according to claim 1, wherein when the device is a lumbar traction device, the at least one diaphragm is three diaphragms.

14. The device according to claim 1, further comprising a wedge system that is only linearly adjustable relative to the carriage adjustable.

15. The device according to claim 13 further comprising a stand slidably mounted to the stationary frame, the stand structured to provide different adjustable angles for the stationary frame relative to a supporting surface.

16. The device according to claim 1, further comprising a locking system which locks the stand at a plurality of different locations on the stationary frame.

17. The device according to claim 16, wherein the locking system includes a spring arm or a hook, and notches or holes associated with a guide on the stationary frame which are structured to accommodate the spring arm or the hook.

18. The device according to claim 14, wherein each wedge of the wedge system is adjustable by a pinch spring arm mechanism.

19. The device according to claim 18, wherein the pinch spring arm mechanism includes at least one ratchet guide and one or more spring arms extending from each wedge and engaging the at least one ratchet guide at different positions via a tabbed or protruding portion.

20. The device according to claim 14, further comprising an anti-rotation device to prevent rotation of wedges of the wedge system.

21. A device comprising a frame forming a housing for accommodating at least one corrugated diaphragm device which is expandable and retractable by application and release of pressure, respectively, the at least one corrugated diaphragm device having a retractable spring force such that, upon release of pressure within the at least one corrugated diaphragm device, the at least one corrugated diaphragm device is configured to retract and move the carriage towards an initial position.

22. The device according to claim 21, wherein;
the frame includes a first frame member and a second frame member, the first frame member includes guides for slidably mounting the moveable carriage,
the second frame member is attached to the first frame member by a fixed hinged member such that the first frame member and the second frame member are foldable between an open position and a closed position,
the first frame member and the second frame member include handle portions such that, when in the folded position, the handle portions are aligned;
the handle portions are formed at least in one of the side and end portions of the first and second frame member.

23. The device according to claim 21, wherein the at least one corrugated diaphragm elastically retracts to a starting position when the pressure is released.

24. The device according to claim 21, wherein the traction system is one of a lumbar traction system and a cervical traction system.

25. The device according to claim 21, wherein the frame is stationary relative to the carriage.

26. A device comprising a stationary frame having a housing for accommodating at least one corrugated diaphragm device which is expandable and retractable by application and release of pressure, respectively, the at least one corrugated diaphragm device being mounted to a moveable carriage which is slidable along the stationary frame between an initial position and an extended position, wherein an overall length of the device remains constant upon the application of pressure to the corrugated diaphragm device and movement of the slidable carriage to any position along the stationary frame.

27. The device according to claim 26, wherein upon release of pressure within the at least one corrugated diaphragm device, the at least one corrugated diaphragm device begins to retract moving the moveable carriage towards an initial position.

28. The device according to claim 26, wherein the at least one corrugated diaphragm device has a spring force to move the moveable carriage towards an initial position upon the release of pressure therein.
